(19) United States
(12) Patent Application Publication
(10) Pub. No.: US 2023/0165503 A1
(43) Pub. Date: Jun. 1, 2023

(54) FLEXIBLE ELECTRICAL MEASUREMENT APPARATUS

(71) Applicant: NEUROCONCISE LTD., Derry (GB)

(72) Inventor: Damien COYLE, Derry (GB)

(21) Appl. No.: 17/754,469

(22) PCT Filed: Oct. 5, 2020
(86) PCT No.: PCT/EP2020/077883

§ 371 Date: Apr. 1, 2022

(30) Foreign Application Priority Data
Oct. 4, 2019 (GB) 1914394.0

(51) Int. Cl. A61B 5/282; A61B 5/00; A61B 5/291

(52) U.S. Cl. CPC A61B 5/282; A61B 5/6803; A61B 5/6814; A61B 5/6835; A61B 5/291

(57) ABSTRACT

An electrical measurement apparatus, comprising, a flexible circuit, the flexible circuit comprising electrical circuitry carried by a flexible substrate, the circuitry comprising one or more electrodes. The electrodes comprise a central portion and a plurality of legs extending radially outwards from the central portion in a spaced apart relationship. At least one electrical contact is located on each one of said plurality of legs; and the one or more electrodes are configured such that the plurality of electrical contacts of the one or more electrodes contact a user's head in use.

### FIELD OF THE INVENTION

This invention relates to a flexible electrical measurement apparatus and in particular a flexible electrical measurement apparatus for performing an electroencephalogram (EEG).

### BACKGROUND TO THE INVENTION

EEG technology is used to measure the electric potentials on the scalp non-invasively. These measurements are traditionally performed using “wet electrodes” to obtain EEG measurements from a user. In these traditional wet electrode arrangements a conductive gel is placed between the sensing apparatus and the user's scalp to enhance conduction of the signal from the scalp to the electrodes of the sensing apparatus by minimising impedances along the way. In this type of wet electrode arrangement the actual sensing apparatus does not necessarily need to touch the scalp due to the conduction enhancing properties of the gel.

Other EEG sensing or measurement devices use what is known as “dry electrodes”. Dry electrodes are more desirable as they remove the necessity to use the conductive gels used for wet electrodes. However dry electrodes are more difficult to implement efficiently as it is a requirement that dry electrodes contact the scalp directly and firmly whilst in use. Furthermore even when the electrode is in contact with the scalp the electrode—scalp impedance can be high and, as a user may move or otherwise cause displacement of the electrode relative to the scalp, intermittent contact can be problematic causing imbalanced impedances between electrodes and time varying impedances resulting in poor signal quality. This may be resolved by the application of pressure to ensure sufficient contact however applying pressure can often cause discomfort for the user and therefore many dry electrodes use soft conductive materials (such as silver doped silicon) or other suitable material. This type of flexible material enables comfort but often results in variable impedances and in some cases poor signal quality.

Furthermore, such dry electrode arrangements are typically made up of a number of individual electrodes which are attached to a cable or wire using, for example, snap connectors or the like which is then in turn connected to a main PCB, normally via wires, where signals are amplified and digitized. These connections between electrode, cabling and PCB serve to complicate manufacturability, may increase impedances or may be associated with noise or interference on the signals at multiple stages of the EEG sensing apparatus.

Therefore it is a desire of the present invention to provide an electrical measurement apparatus suitable for EEG measurements which overcomes the above deficiencies.

### SUMMARY OF THE INVENTION

Accordingly the present invention provides an electrical measurement apparatus, comprising: a flexible circuit, the flexible circuit comprising electrical circuitry carried by a flexible substrate, said circuitry comprising one or more electrodes, wherein said electrodes comprise a central portion and a plurality of legs extending radially outwards from the central portion in a spaced apart relationship, wherein at least one electrical contact is located on each one of said plurality of legs; and wherein said one or more electrodes are configured such that the plurality of electrical contacts of the one or more electrodes contact a user's head in use.

Preferably, wherein said flexible circuit comprises a plurality of the electrodes integrally connected to the processing circuitry.

Ideally, wherein the electrical circuitry extends within the flexible substrate to the tips of each of the plurality of legs of the electrode.

Ideally, wherein said electrodes comprise a support structure which is coupled to the central portion and/or the one or more legs.

Preferably, wherein the support structure comprises a main body with a plurality of limbs extending radially outwards from the main body in a spaced apart relationship.

Ideally, wherein the support structure is substantially domed shaped.

Preferably, wherein the cross section of the support structure, extending from a distal end of a first limb to a distal end of an opposing second limb traversing the main body, is substantially arcuate.

Ideally, wherein the plurality of legs each comprise an aperture located at or towards the distal end of each leg.

Preferably, wherein the protrusions are located at or towards the proximal ends of the plurality of limbs.

Ideally, wherein the protrusions are located on a first surface of the support structure.

Preferably, wherein the plurality of legs are folded laterally about the plurality of limbs, wherein the apertures of the plurality of legs align and engage with the plurality of protrusions to couple the plurality of legs to the support structure.

Ideally, wherein the central portion is located adjacent to or in contact with a second surface of the support structure when the plurality of legs are coupled to the support structure.

Preferably, wherein the portion of each of the legs which is folded laterally about the tips of the limbs defines at least part of the electrical contact for each leg.

Ideally, wherein the support structure is coupled to the central portion and/or the one or more of the plurality of legs by an adhesive or heat-sealing.

Preferably, wherein said electrode further comprises a cover which is coupled to the support structure.

Ideally, wherein said cover is coupled to the first surface of the support structure.

Preferably, wherein said cover comprises a plurality of recesses on its underside which are configured to engage with the protrusions formed on the first surface of the main body such as to couple the cover to the support structure.

Ideally, wherein the first surface of the main body comprises a central protrusion which is configured to engage with a co-operable recess provided on the underside of the cover such as to couple the cover to the support structure.

Preferably, wherein the central portion, the support structure and the cover each comprise a respective central aperture through which a retaining member, typically comprising a pin, is inserted to couple the support structure and cover or the support structure, cover and central portion together.

Ideally, wherein said processing circuitry is located in a portion of said flexible circuit that is spaced apart from the respective portions that carry said electrodes.

Preferably, wherein the support member is made of rubber.

Ideally, wherein the cover is made of rubber and/or plastic.

Preferably, further comprising means for receiving a fluid, typically comprising a conductive gel, and a dispersion member which is configured to distribute the fluid received along the legs towards the electrical contacts.

Ideally, wherein the means for receiving a fluid comprises a retaining member which is configured to couple the dispersion member and support structure together, wherein the retaining member comprises a hollow elongate member defining a substantially tube like structure.

Preferably, wherein the hollow elongate member defines a channel for receiving the fluid.

Ideally, wherein the dispersion member comprises one or more outlets which are in fluid communication with the channel when the dispersion member is coupled to the retaining member.

Preferably, wherein the dispersion member is shaped and dimensioned to direct fluid which egress from the outlets towards the electrical contacts.

A second aspect of the invention provides a headgear apparatus comprising the electrical measurement apparatus as claimed in any of claims 1 to 24.

Ideally, wherein the electrical measurement apparatus is integrally coupled to the headgear apparatus.

A third aspect of the invention provides a method of manufacturing the electrical measurement apparatus as claimed in any of claims 1 to 24, comprising:

Providing the flexible circuit comprising electrical circuitry carried by the flexible substrate, said circuitry comprising the one or more electrodes;

Coupling the support structure to the flexible circuit.

Preferably, further comprising coupling the cover to the flexible circuit and/or support structure.

Ideally, wherein coupling the support structure to the flexible circuit comprises folding one or more parts of the flexible circuit about the support structure such as to couple the flexible circuit and support structure together.

Ideally, coupling the support structure to the flexible circuit comprises engaging respective engagement means provided on the support structure and flexible circuit respectively.

Optionally, coupling the support structure to the flexible circuit may comprise using adhesive or heat sealing.

Preferably, coupling the cover to the flexible circuit and/or support structure comprises engaging respective engagement means provided on the cover and support structure and/or flexible circuit respectively.

Preferably, providing the flexible circuit comprising electrical circuitry carried by the flexible substrate, said circuitry comprising the one or more electrodes may further comprise providing the flexible circuit upon a guide means which is typically configured to position the flexible circuit for alignment with the support structure prior to coupling the support structure to the flexible circuit.

An electrical measurement apparatus, comprising: a flexible circuit, the flexible circuit comprising electrical circuitry carried by a flexible substrate, said circuitry comprising one or more electrodes integrally connected to processing circuitry, wherein said electrodes comprise a central portion and a plurality of legs extending radially outwards from the central portion in a spaced apart relationship, wherein at least one electrical contact is located on each one of said plurality of legs; and wherein said one or more electrodes are configured such that the plurality of electrical contacts of the one or more electrodes contact a user's head in use.

### BRIEF DESCRIPTION OF THE DRAWINGS

The invention will now be described with reference to the accompanying drawings by way of example in which like numerals are used to denote like features and in which:

FIG. 1 shows a top plan view of the electrical measurement apparatus;

FIG. 2 shows a top plan view of an electrode of the electrical measurement apparatus;

FIG. 3 illustrates a perspective view of an electrode positioned on a guide means;

FIG. 4 illustrates a perspective view of the electrode positioned on the guide means and a support structure being coupled thereto;

FIG. 5 illustrates a perspective view of the electrode positioned on the guide means and the support structure coupled thereto;

FIG. 6 illustrates a perspective view of the electrical measurement apparatus in particular, the electrode having the support structure coupled thereto;

FIG. 7 illustrates a perspective view of the electrical measurement apparatus in particular showing a cover being coupled thereto;

FIG. 8 illustrates a perspective view of the electrical measurement apparatus in particular showing the cover coupled to the support structure;

FIG. 9 illustrates a perspective view of the electrical measurement apparatus in particular showing a second embodiment of the cover being coupled to the support structure;

FIG. 10 illustrates a perspective view of the electrical measurement apparatus in particular showing the second embodiment of the cover coupled to the support structure;

FIG. 11 shows an exploded view of the electrical measurement apparatus in particular showing the arrangement of the electrical circuit, support structure, cover and an attachment means for coupling the electrode to a piece of headgear;

FIG. 12 is a sectional view of the electrical measurement apparatus in particular showing the coupled arrangement of the electrical circuit, support structure, cover and an attachment means for coupling the electrode to a piece of headgear;

FIG. 13 is a perspective view of the electrical measurement apparatus in particular showing the coupled arrangement of the electrical circuit, support structure, cover and an attachment means for coupling the electrode to a piece of headgear;

FIG. 14 shows a bottom perspective exploded view of the electrical measurement apparatus, in particular showing a third embodiment of the cover;

FIG. 15 shows a front perspective exploded view of the electrical measurement apparatus, in particular showing the third embodiment of the cover;

FIG. 16 shows a front perspective exploded view of the electrical measurement apparatus, in particular showing a fourth embodiment of the cover;

FIG. 17 shows a front perspective view of the electrical measurement apparatus, in particular showing the fourth embodiment of the cover;

FIG. 18 shows a rear perspective view of the electrical measurement apparatus, in particular showing the fourth embodiment of the cover;

FIG. 19 shows a side perspective view of the electrical measurement apparatus, in particular showing the fourth embodiment of the cover;

FIG. 20 shows a top perspective view of an alternative embodiment of the electrical measurement apparatus;

FIG. 21 shows a bottom perspective view of the alternative embodiment of the electrical measurement apparatus;

FIG. 22 shows a further top perspective view of the alternative embodiment of the electrical measurement apparatus;

FIG. 23 shows a further bottom perspective view of the alternative embodiment of the electrical measurement apparatus;

FIG. 24 shows an exploded view of the alternative embodiment of the electrical measurement apparatus;

FIG. 25 shows a further front perspective view of the alternative embodiment of the electrical measurement apparatus;

FIG. 26A shows a top plan view of the alternative embodiment of the electrical measurement apparatus;

FIG. 26B shows a sectional view of the alternative embodiment of the electrical measurement apparatus;

FIG. 27 shows a further bottom perspective view of the alternative embodiment of the electrical measurement apparatus;

FIG. 28 shows a further exploded view of the alternative embodiment of the electrical measurement apparatus;

FIG. 29 is a rear perspective view of the electrical measurement apparatus worn on a user;

FIG. 30 is a front perspective view of a flexible circuit;

FIG. 31 is a front perspective view of the flexible circuit;

FIG. 32 is a front perspective view of the flexible circuit coupled to a stiffener;

FIG. 33 is a rear perspective view of an alternative embodiment of the stiffener coupled to the flexible circuit; and

FIG. 34 is a perspective view of the overmoulded flexible circuit.

### DETAILED DESCRIPTION

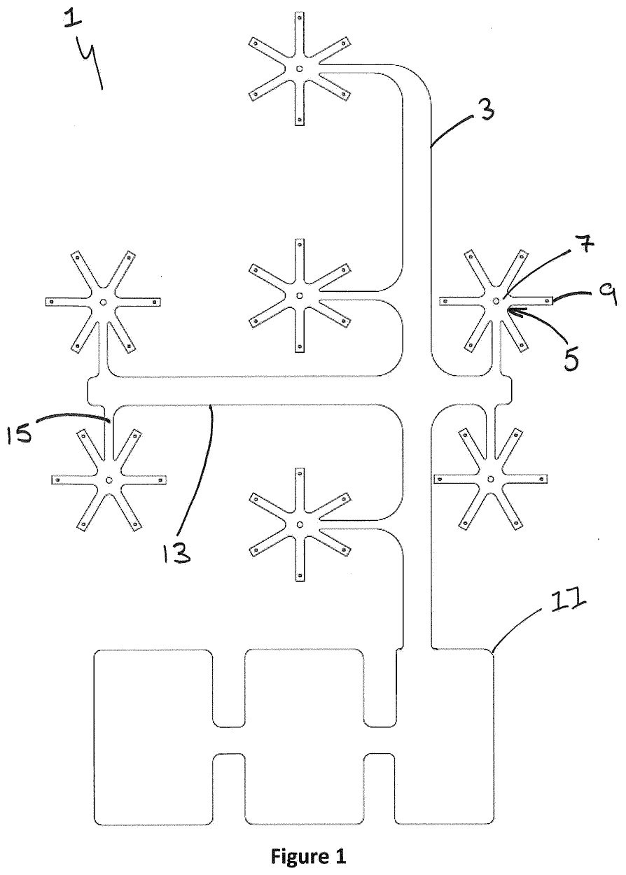

Referring now to the drawings, in particular FIG. 1 thereof, there is shown generally indicated by the reference numeral 1 an electrical measurement apparatus. The electrical measurement apparatus 1 comprises a flexible circuit 3, the flexible circuit comprising electrical circuitry carried by a flexible substrate, said circuitry comprising one or more electrodes 5. The electrical circuitry typically comprises processing circuitry which may be integrally connected to the electrodes 5. The electrodes 5 comprise a central portion 7 and one or more legs 9, typically a plurality of legs 9, extending radially outwards from the central portion 7 in a spaced apart relationship. Typically at least one electrical contact is located on each one of the plurality of legs 7. The one or more electrodes 5 are configured such that the plurality of electrical contacts of the one or more electrodes 5 contact a user's head in use. The flexible circuit 3 preferably comprise a plurality of the electrodes 5. Further the electrical circuitry ideally extends within the flexible substrate to the tips of each of the plurality of legs 9 of the electrode 7.

The flexible circuit 3 typically comprises a number of different sections within which like components are arranged in close proximity. The processing circuitry is typically located in a portion of said flexible circuit 3 that is spaced apart from the respective portions that carry said electrodes 5. For example as shown in FIG. 1 the flexible circuit 3 may comprise a posterior portion 11, along with an anterior portion 13. Wherein the posterior portion 11 typically comprises the electrical components such as processing means e.g. CPU, amplifiers etc. which are operable to receive and process information, in the form of electrical signals, received from the electrodes 5. In-use, such as where the electrical measurement apparatus 1 may be mounted on a user's head, either directly or via incorporation within some form of headgear or the like, the posterior portion 11 is typically located at or extending towards the back of the user's head. The anterior portion 13, as can also be seen in FIG. 1 is coupled, in integral fashion, to the posterior portion 11, the anterior portion 13 comprising one or more elongate sections to which the one or more electrodes 5 branch off therefrom, the electrodes 5 comprising a stem portion 15 from which the electrodes branch off of the anterior portion 13. In-use such as where the electrical measurement apparatus 1 may be mounted on a user's head, either directly or via incorporation within some form of headgear or the like as mentioned previously, the anterior portion 13 is typically located at or extending towards the front of the user's head.

Advantageously all of the electrical circuitry from the electrode 5 to the processing circuitry is embedded within a single integral printed circuit (PCB) which has a number of further benefits such as the ease of manufacturability and the lack of connections present between respective parts such as is common in prior art arrangements where the electrodes are coupled to cable via a clip or other connector to a cable or wire which is in turn connected via similar means to a processing circuit. In comparison the presently described electrical measurement apparatus therefore is further advantageous as the lack of external connection points reduces the potential for water ingression and potentially most advantageously the continuous flexible circuit extending from the tip of the electrodes 5 to the processing circuitry, typically located within the posterior portion 11 of the electrical circuit 3, means that electrical noise interference and impedance variations are reduced in the electrical signals obtained using the electrical measurement apparatus 1.

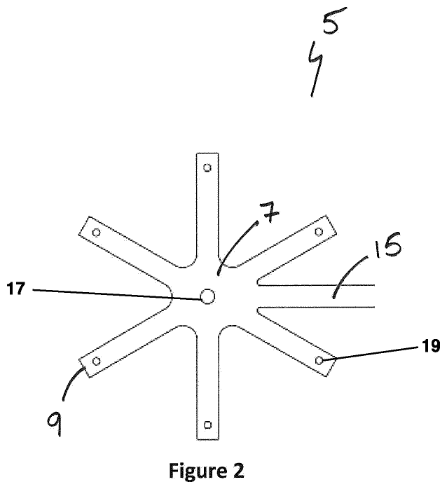

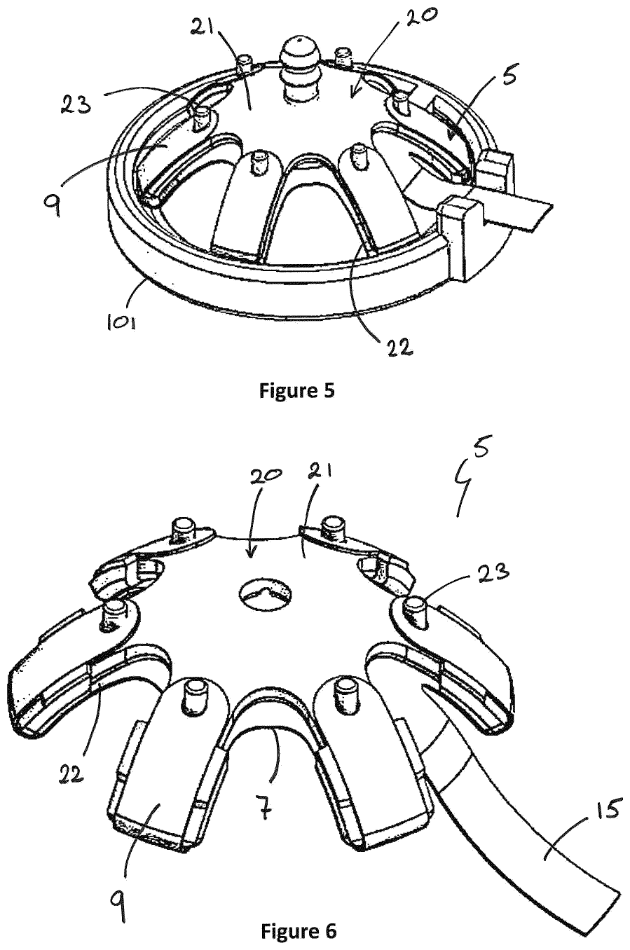

Referring now to FIG. 2, there is shown a top plan view of the electrode 5. The electrode 5, as mentioned previously, comprises the central portion 7 and the plurality of legs 9 extending radially outwards from the central portion 7 in a spaced apart relationship. FIG. 5 also shows the stem portion 15 by which the electrode 5 is integrally coupled to the anterior portion 13 of the flexible circuit 3. The plurality of legs 9 each typically comprise an aperture 19 which is preferably located at or towards the distal end of each leg 9. Additionally the central portion 7 may also comprise a central aperture 17.

The electrodes 5 preferably further comprise a support structure 20, which is configured to provide additional support to the portions of the flexible circuit making up the electrode when it is coupled thereto. To this end the support structure 20 is typically coupled to the central portion 7 and/or the one or more legs 9 of the electrode 5. The support structure 20 ideally comprises a main body 21 with a plurality of limbs 22 extending radially outwards from the main body 2 in a spaced apart relationship. The support structure 20 is typically substantially dome-shaped such that a cross section of the support structure 20, extending from a distal end of a first limb 22 to a distal end of an opposing second limb 22 traversing the main body 21 is substantially arcuate. As mentioned the support structure 20 is preferably coupled at least to the plurality of legs 9, to this end the support structure preferably comprises a plurality of protrusions 23 which are configured to engage and retain the plurality of legs 9 relative to the support structure 20. The protrusions 23 are typically located on a first surface 24 of the support structure 20, typically at least in part upon the limbs 22. Ideally a protrusion 23 is located upon each of the limbs 22 typically at or towards the proximal ends of the limbs 22.

The plurality of legs 9 are coupled to the plurality of limbs 22 at least by engagement of the protrusions 23 of the limbs 22 with the apertures 19 of the legs 8. To this end the number of limbs 22 preferably corresponds to the number of legs 9. When the plurality of legs 9 are coupled to the plurality of limbs 22 as described the plurality of legs 9 define a lateral fold about the width of each of the plurality of limbs 22 such that the longitudinal length of each leg 9 extends substantially along the longitudinal length of the first and second opposing surfaces of each limb 22 with the lateral fold extending at least in part across the tip of the limbs 22. Ideally the central portion 7 is located adjacent to or in contact with a second surface 25 of the support structure 20 when the plurality of legs 9 are coupled to the support structure 20. The support structure 20 is made of a flexible material such as rubber, plastic or any other suitable material.

The plurality of legs 9 are each folded laterally or crosswise about the width of the plurality of limbs 22 such that the apertures 19 of the legs 9 align and engage with the plurality of protrusions 23 located on the limbs 22 such as to couple the plurality of legs 9 to the limbs 22 and to the support structure 20. Preferably the lateral fold comprising the portion of each of the legs 9 which is folded laterally about the tips of the limbs 22 defines at least part of the electrical contact for each leg 9. Optionally the coupling of the plurality of legs 9 to the plurality of limbs 22 may comprise an adhesive or heat-sealing to further enhance the coupling.

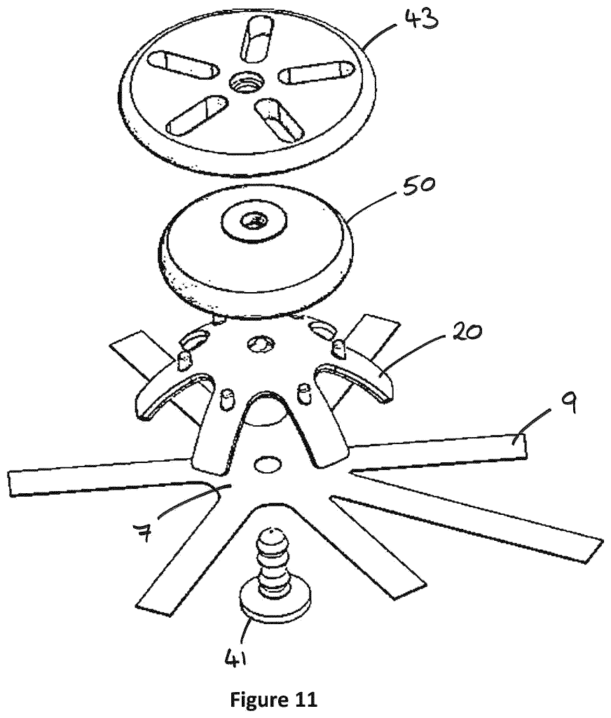

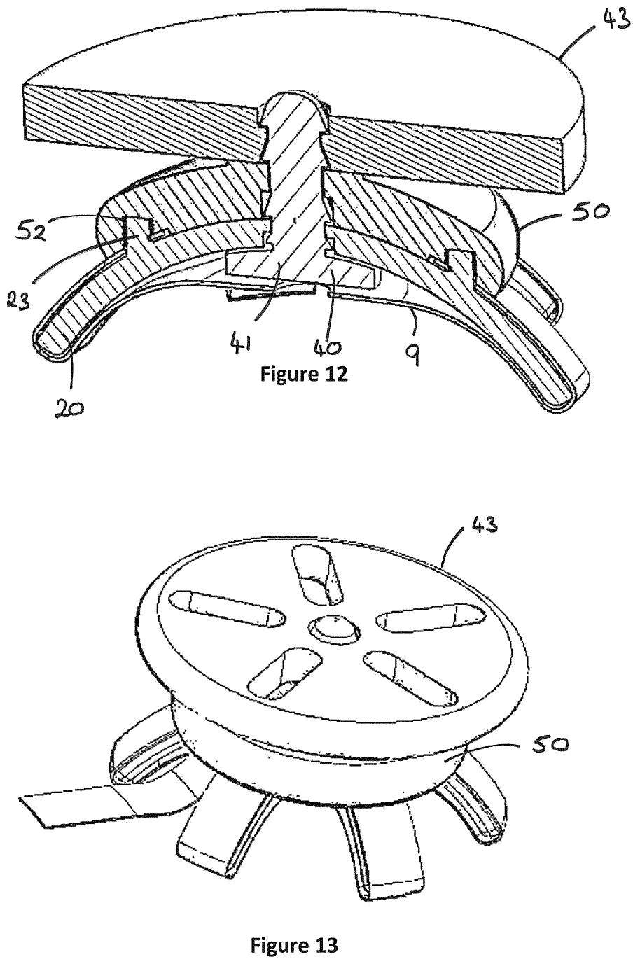

The electrode 5 may further comprise a cover 30 or the like which may be coupled to the support structure 20. The cover 30 is configured to provide protection for the electrode 5, in particular the legs 9 and the coupling between the support structure 20 and the legs 9. The cover 30 is typically coupled to the first surface 24 of the support structure 20. In one embodiment the cover 30 comprises a plurality of recesses 52 on its underside which are configured to engage with the protrusions 23 formed on the first surface 24 of the main body 21 such as to couple the cover 30 to the support structure 20. Additionally or alternatively the first surface 24 of the main body 21 may comprise a central protrusion 26, typically located on the first surface 24 thereof, which is configured to engage with a co-operable recess provided on the underside of the cover 30 such as to couple the cover 30 to the support structure 20. In an alternative embodiment such as that shown in FIGS. 11 to 15, the central portion 7, the support structure 20 and the cover 30 may each comprise a respective central aperture through which a retaining member 40, typically comprising a pin, is inserted to couple the support structure 20 and cover 30 or the support structure 20, cover 30 and central portion 7 together.

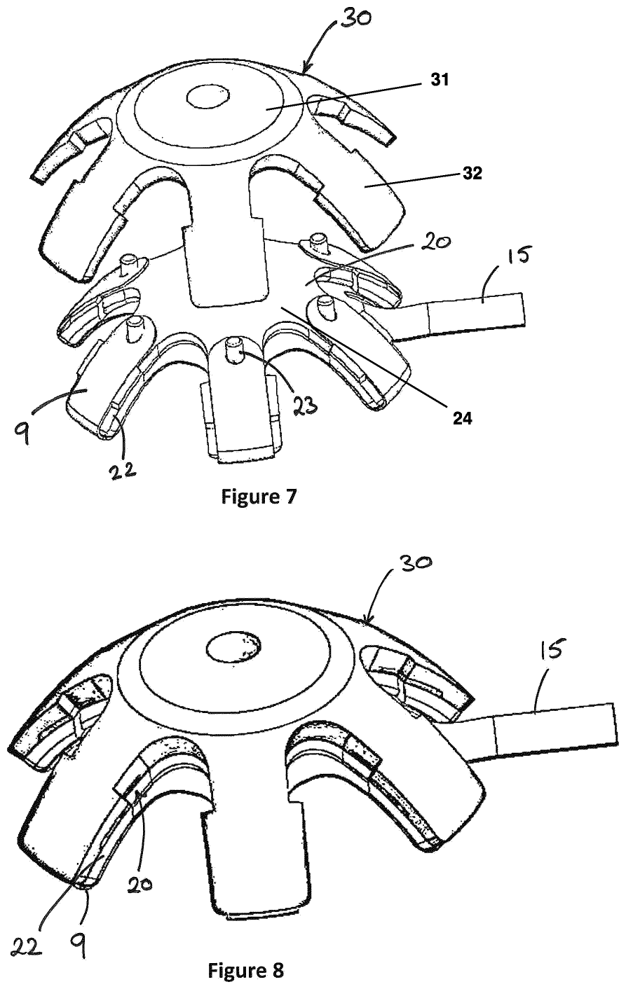

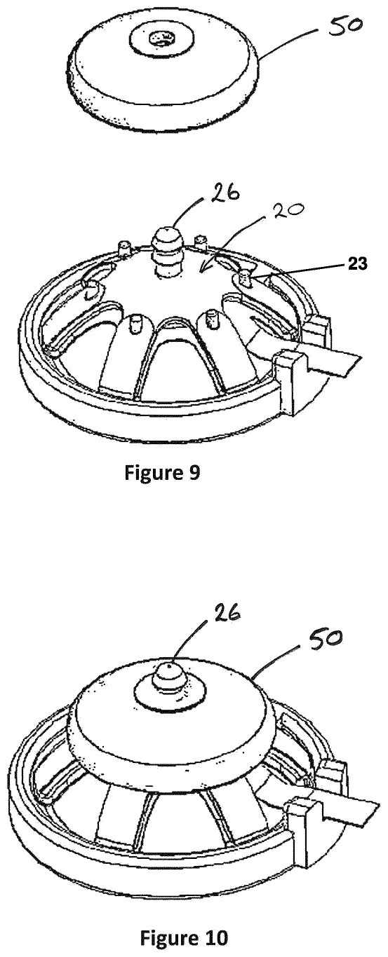

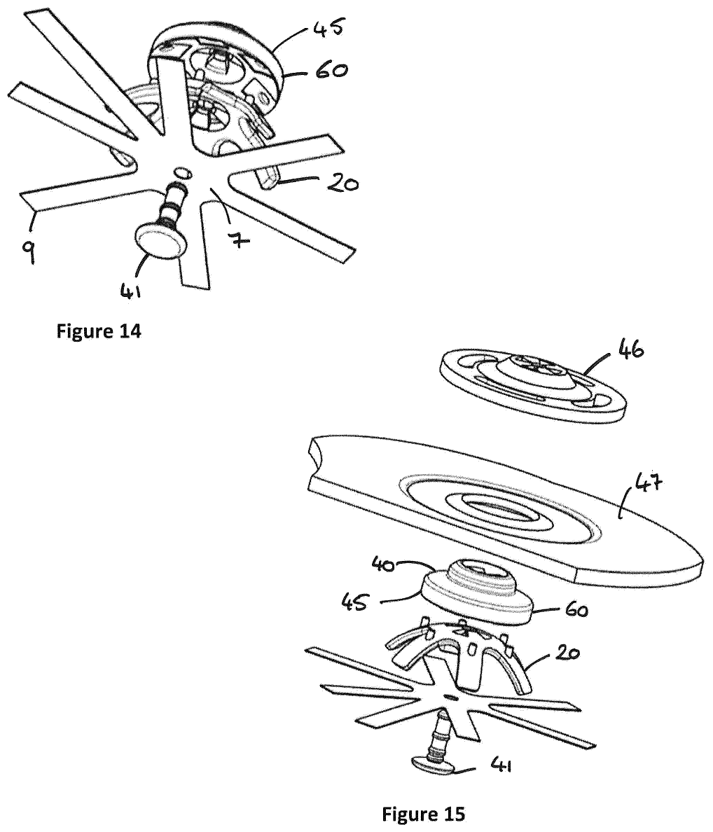

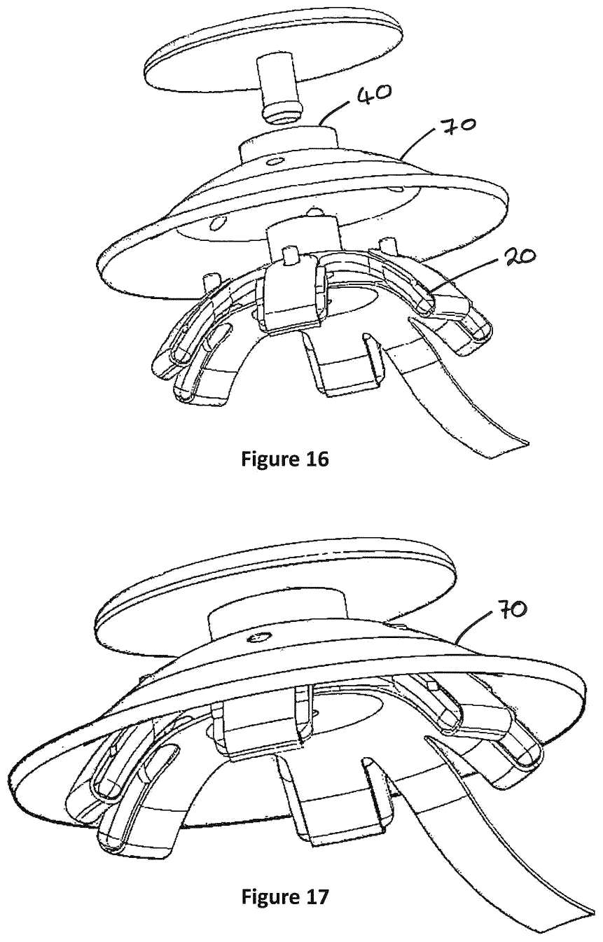

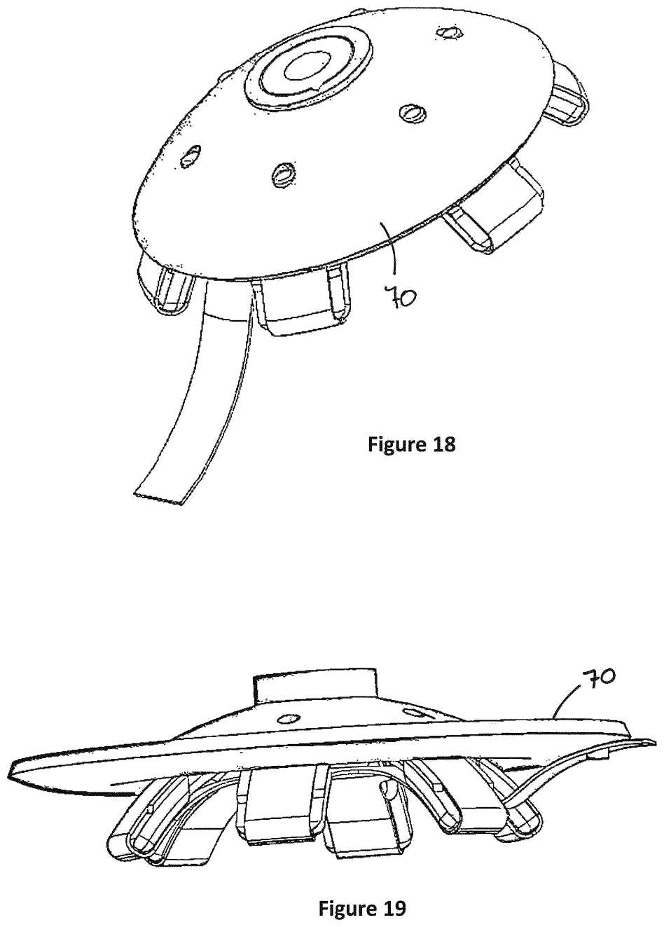

A first embodiment of the cover 30 is shown at FIGS. 7 to 8 wherein the cover 30 may substantially conform to the shape and dimensions of the support structure 20 in that it comprises a cover main body 31 and cover limbs 32 which align with the main body 21 and limbs 22 when the cover 30 is coupled to the support structure such as is shown in FIGS. 7 and 8. In an alternative second embodiment such as that shown in FIGS. 9 to 13 the cover 50 may be substantially disc shaped. A third alternative embodiment of the cover 60 is shown in FIGS. 14 and 15, wherein the cover 60 is also substantially disc-shaped. Further alternatively in a fourth alternative embodiment the cover 70 may be substantially dome-shaped such as that shown in FIGS. 16 to 19. The cover 30, 50, 60 may further comprise an attachment means 40 for coupling the electrical measurement apparatus 1 to a headgear such as a cap or hat or the like. For example the attachment means 40 may comprise a pin or peg which is configured to engage with a hole provided in a button. Alternatively or additionally where the central portion 7, the support structure 20 and the cover 30 are coupled together by a coupling member 41 such as a pin or the like which extends through the central aperture of the central portion 7, the support structure 20 and the cover 30, the coupling member may extend beyond the central aperture of the cover 30 such as to define the attachment means 40, wherein the attachment means 40 is configured to engage with a co-operable attachment means provided on the headgear such as central hole formed in a button or the like such as is shown in FIGS. 11 to 13. Further alternatively the attachment means 40 may comprise an orifice or the like formed in the cover 30 which is operable to engage with a member formed in the headgear.

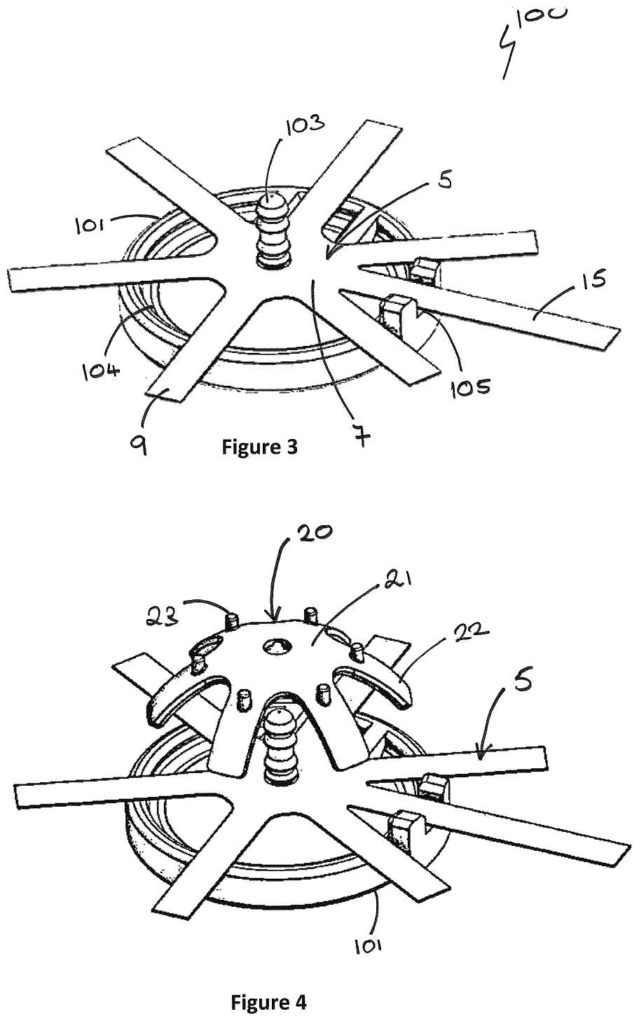

Referring now to the drawings and in particular FIG. 3, there is shown generally indicated by the reference numeral 100 a first stage in the coupling of at least the support structure 20 to the electrode 5 of the electrical measurement apparatus 1. The electrode 5 is located on a guide means 101 such as a jig such that the central aperture formed in the central portion 7 aligns and engages with a central retaining member 103 on the guide means 101. The guide means 101 is typically substantially circular in shape with a portion of the guide means 101 defining a channel 105 at a point along the periphery of the guide means 101 within which the stem portion 15 of the electrode 5 may be received and retained when the central portion 7 is engaged on the central retaining member 103. The guide means 101 further typically comprises upstanding central portion 106 which is surrounded by an interior groove 104 which extends around the inner periphery of the guide means 101, typically around the entire circumference. Once the electrode 5 is thus positioned upon the guide means 101, the support structure 20 may be positioned on top of the central portion 7, wherein the main body 21 of the support structure 20 comprising a central aperture may align with the central retaining member 103. When the support structure 20 is mounted upon the central retaining member 103, on top of the central portion 7, the limbs 22 are aligned with the legs 9 and a downward pressure is applied to the support structure 20 such that the legs 9 are laterally fold about the limbs 22. Upon application of the downward pressure, at least the tips of the limbs 22 are pressed into the groove 104, with the legs 9 being guided by the interior walls of the guide means 101. Upon folding of the legs 9 about the limbs 22, the apertures of the legs 9 are aligned with the protrusions on the limbs 22 such as to couple the legs to the limbs such as is illustrated at FIG. 5. Additionally or alternatively an adhesive may be placed on the legs or the limbs to couple the legs 9 to the limbs 22. Further additionally or alternatively the legs 9 may be heat sealed to the limbs 22 or coupled thereto in any suitable manner. Subsequent to the coupling of the plurality of legs 9 to the plurality of limbs the electrical measurement apparatus 1 may be removed from the guide means 101, typically by being lifted off of the upstanding central portion 106.

The electrode 5 comprising the coupled support structure 20 is shown at FIG. 6. Advantageously the additional structural support provided by the support structure 20 allows the limbs 22 and coupled legs 9 to more easily penetrate the user's hair such as to affect efficient contact between the electrode 5 and the user's scalp. The cover 30 is typically provided to at least provide protection to the flexible circuit 3, a portion of which is typically exposed upon the first surface 24 of the support structure 20. FIG. 7 illustrates the first embodiment of the cover 30 which substantially mirrors the overall shape and dimensions of the support structure 20. To this end the cover 30 in this first embodiment comprises the cover main body 31 and cover limbs 32. Preferably the number of cover limbs 32 corresponds to the number of limbs of the support structure 20 and incidentally ideally the number of legs 9. In this first embodiment of the cover 30 it typically comprises a plurality of recesses (now shown) on its underside which are configured to engage with the protrusions 23 formed on the first surface 24 of the main body 21 of the support structure 20 such as to couple the cover 30 to the support structure 20. This can be seen in FIGS. 7 and 8 where in FIG. 7 the cover 30, in particular cover legs 32 thereof, are aligned on the same vertical plane such that upon movement of the cover 30 and/or support structure 20 towards one another the protrusions 23 provided on the first surface 24 of the support structure 20 engage the recesses provided upon the underside of the cover 30.

Additionally or alternatively an adhesive may be used to couple the cover 30 to the support structure 20. The cover 30 acts to prevent dirt and debris reaching exposed portions of circuitry 3 located on the first surface 24 of the main body 21, however it should be understood that in this embodiment the cover does not extend along the limbs 22 to such an extent such as to obscure the portions of electrical circuitry 3 exposed around the tips of the limbs 22, nor does it extend over the second surface 25 of the main body 21 such as to obscure the central portion 7, in this manner the electrical circuit 3 of the electrodes 5 can contact the user's scalp when the electrical measurement apparatus 1 is in-use and mounted upon or provided in contact with the user's scalp.

Referring now to FIGS. 9 to 13 there is shown the second embodiment of the cover 50. The second embodiment of the cover 50 comprises a substantially disc-shaped element. Further in this embodiment the support structure 20 comprises one more protrusions 23, 26 which are configured to engage with one or more recesses 52 provided upon the cover 50, typically upon the underside thereof. For example the cover 50, similar to the first embodiment, may comprise a plurality of recesses within which the protrusions 23 of the support structure may engage to couple the cover 50 to the support structure 20. Additionally or alternatively the support structure 20 comprises a central protrusion 26 which is configured to engage with a central aperture 51 formed in the cover 50 such as to couple the cover 50 to the support structure 20. Further additionally or alternatively the cover 50 can be coupled to the support structure 20 by an adhesive or heat sealing or any other suitable coupling means. This as is shown in FIGS. 9 and 10 can be performed whilst the electrode 5 is mounted or otherwise coupled to the guide means 101.

As mentioned in relation to the second embodiment of the cover 50, the main body can comprise the central protrusion 25 which can extend through the cover to such an extent as to define the attachment means 40 i.e. a significant length of the central protrusion 25 extends through and out of the central aperture of the cover such that a co-operable attachment means provided on a piece of headgear or the like can engage and retain or be retained by the central protrusion 25. In a preferred embodiment, which is applicable to each of the embodiments of the cover 30, 50, as described previously each of the central portion 7, the support structure 20, and the cover 30, 50 may each comprise a central aperture through which the coupling member 41 may be inserted such as to couple the central portion 7, the support structure 20, and the cover 30, 50 together as can be seen in FIGS. 11 to 15. Additionally the coupling member 41 extends through each of the central portion 7, the support structure 20, and the cover 30, 50 and further to such an extent that the portion of the coupling member 41 extends beyond the cover 30, 50 defines the attachment means 40, which is configured to engage with the co-operable attachment means 43 of a piece of headgear, which may comprise a button or the like such as is shown in FIGS. 11 to 13.

Referring now to FIGS. 14 and 15 there is shown the third alternative embodiment of the cover 60. In this embodiment the cover 60 comprises a substantially disc-shaped element, the top surface of which defines a first part of a co-operable attachment means 45 which is co-operable with a corresponding other second part 46 typically provided upon the headgear or the like to which the electrode 5 is to be coupled. The first and second parts 45, 46 typically comprise corresponding male and female members which are configured to engage with one another. A spacer element 47 or the like may be provided between the first and second parts 45, 46 the spacer element comprising a central aperture which allows the corresponding male and female members to engage with one another there through.

Referring now to FIGS. 16 to 19 there is shown the fourth embodiment of the cover 70. In this embodiment the cover 70 is substantially dome-shaped. The cover 70 comprises an attachment means 40 comprising a female portion which is engageable with a corresponding male portion provided on the headgear or the like.

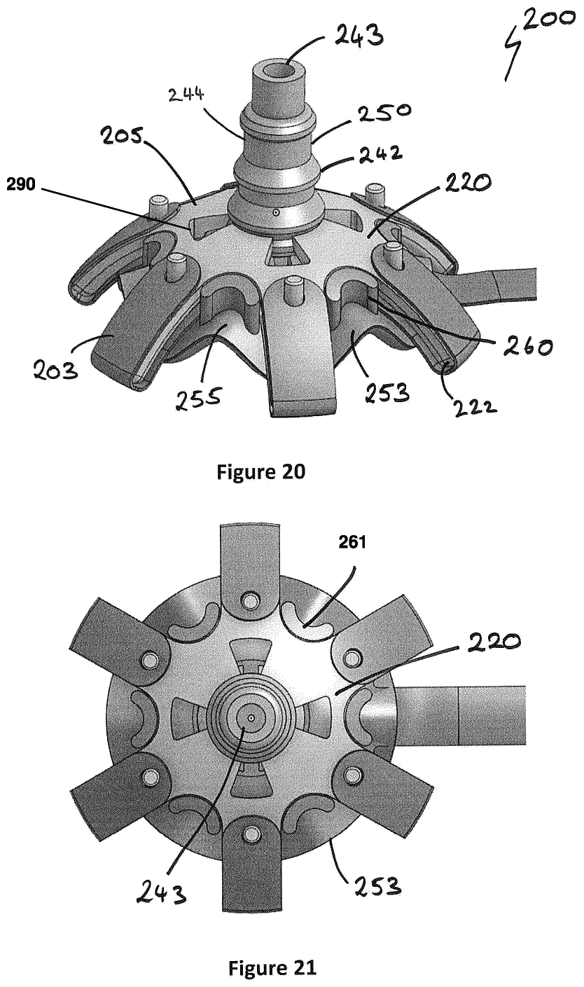

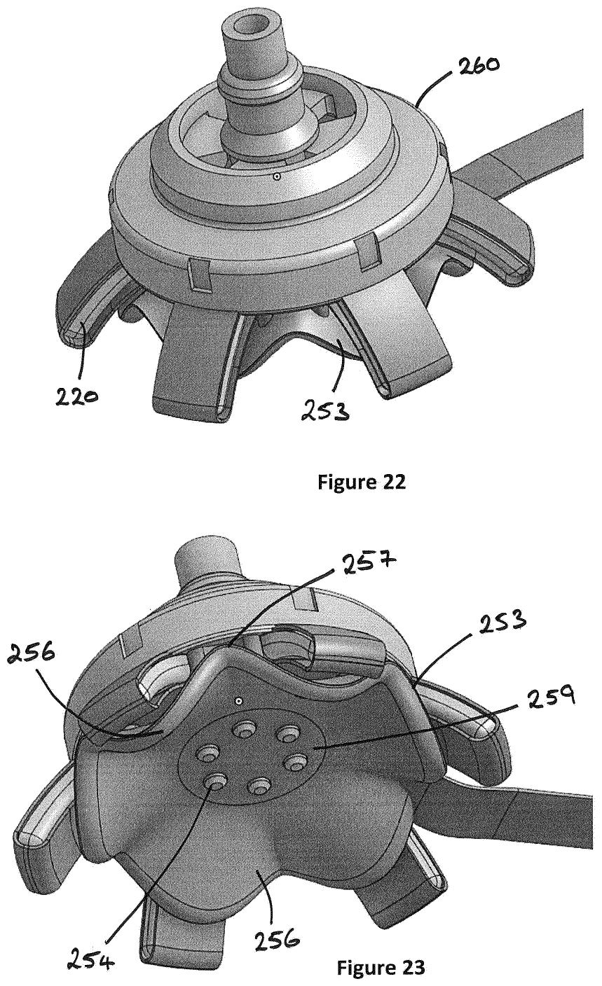

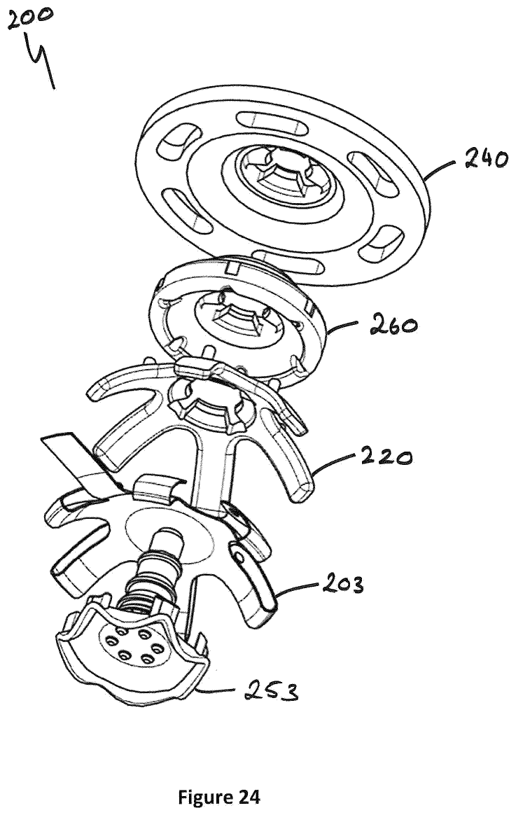

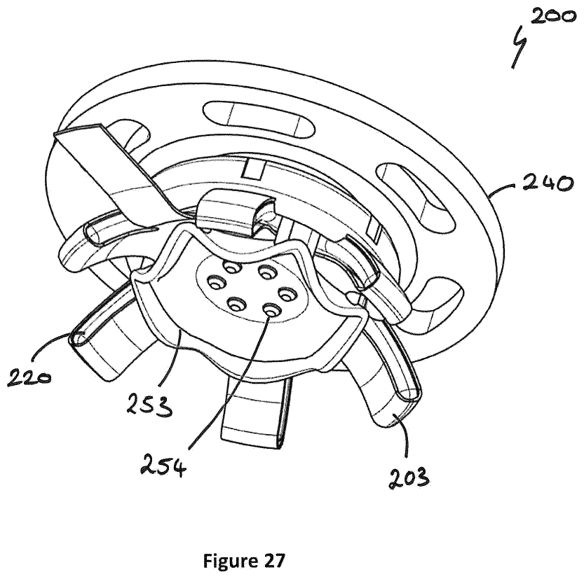

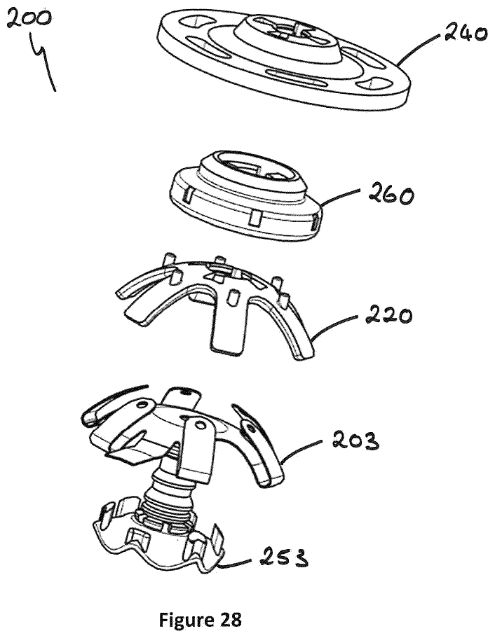

Referring now to FIGS. 20 to 27 there is shown an alternative embodiment of the electrical measurement apparatus indicated generally by the reference numeral 200. In this embodiment the electrode 205 comprises means for receiving a fluid 250, typically comprising a conductive gel or the like, into the electrode 205, the electrode 205 further comprises a dispersion member 253 which is configured to distribute the fluid received via the means for receiving a fluid 250, to this end the dispersion member 253 is shaped and dimensioned to distribute the fluid received across at least one surface, wherein said surface is that which is arranged to contact the user's head in-use. The support structure 220 and/or cover 260 may comprise one or more apertures 290 for allowing any excess fluid or fluid disposed upon the electrode 205 to flow through the electrical measurement apparatus towards the dispersion member 253.

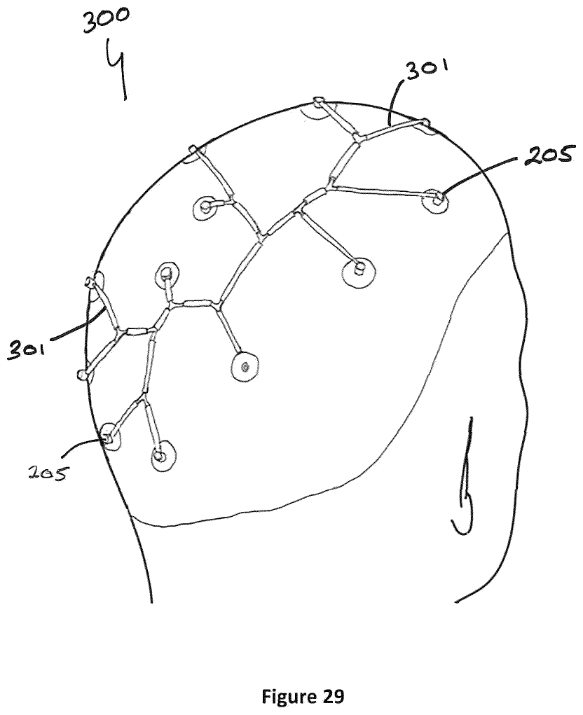

Referring to FIGS. 20 and 21 in particular, the flexible circuit 203 is shown coupled to the support structure 220 and the dispersion member 253 is further coupled to the support structure 220 by the means for receiving a fluid 250. The means for receiving a fluid 250 comprises a retaining member 244 which extends through an aperture, typically a central aperture, of the flexible circuit 203 and support structure 220 to couple the dispersion member 253 and support structure 220 together. To this end the retaining member 244 is configured to couple to a first surface 255 of the dispersion member 253, the first surface being the surface in contact with and/or closest to the underside of the support structure 220 when the dispersion member 253 and support structure 220 are coupled together. As shown in FIG. 20 the retaining member 244 comprises a hollow elongate member defining a substantially tube like structure. The retaining member 244 preferably further comprises one or more outwardly extending lips 242 which extend around the circumference of the retaining member, the one or more outwardly extending lips 242 are spaced apart from one another along the length of the retaining member 244. The one more one or more outwardly extending lips 242 are configured to engage with corresponding recesses (not shown) provided in the support structure 220 and/or cover 260. The hollow elongate member defines a channel 243 through which a fluid may be injected. The channel 243 may be coupled to a fluid delivery system 300 such as that shown in FIG. 29. A problem with the electrical measurement apparatus 1, 200 which comprises multiple electrodes 205 when used with conductive gel/fluids (water or saline solution) is that each electrode 205 has to be injected or dosed with gel/fluid individually. This can be very time consuming, particularly when many electrodes 205 are in use. The channel 243 of each of the electrodes 205 of the electrical measurement apparatus 200 may be removably coupled to the fluid delivery system 300. The fluid delivery system 300 comprises a plurality of hoses 301 which are coupleable to the corresponding plurality of electrodes 205, the hoses being removably coupleable at one end to the channel 243 of each of the electrodes 205, the hoses 301 sharing a common entry point at their other end (not shown) which allows for fluid/gel to be injected at the common entry point for delivery to multiple or preferably all of the electrodes 205 of the electrical measurement apparatus 200. This allows for easy application of gels/fluids and topping up of gels when the cap is in use for longer durations.

The dispersion member 253 comprises one or more outlets 254 which are provided on a second surface 256 of the dispersion member 253. When the dispersion member 253 is coupled to the retaining member 244 the channel 243 of the retaining member 241 is in fluid communication with the one or more outlets 254, such that when fluid is received within the channel 243 it is operable to flow through the channel 243 to the dispersion member 253 and out of the outlets 254 formed on the second surface 256 thereof. It should be understood that the first and second surfaces of the dispersion member 253 comprise the obverse and reverse sides of the dispersion member 253. The dispersion member 253 is preferably shaped and dimensioned such as to distribute the fluid substantially evenly across the second surface thereof. To this end, as shown in FIGS. 20 and 23 the dispersion member 253 is substantially flower-shaped, having a substantially flat centre portion 259, upon which the outlets 254 are typically located, and a peripheral edge which typically defines a plurality of troughs 256 and ridges 257, the ridges 257 extending between the limbs of the support structure 222 and the troughs 256 extending at least in part along the length of the limbs 222 such as to define a plurality of pathways, wherein the curved shape of the troughs 256 is such that fluid egressing from the outlets 254 is operable to flow, at least in part, along the pathways defined by the troughs 256 towards the ends of the limbs 222 and the tips of each of the plurality of legs 209 of the electrode 205.

Ideally the dispersion member 253 is configured to couple to the cover 260. To this end the dispersion member 253 comprises one or more protrusions 261, typically formed on the first surface thereof, which are configured to engage with corresponding recesses formed on the cover 260 such that the cover 260 is coupled to the dispersion member 253. The protrusions are typically formed on the first surface upon one or more of the ridges 257. Further the protrusions 261 are configured to direct any fluid received on the first surface of the dispersion member 253 towards the troughs 256 and away from the ridges 257. To this end the protrusions 261 are typically located at the apex of each of the ridges 257 and are shaped to direct the flow of fluid towards the troughs 256 accordingly. For example as shown in FIGS. 20 and 21, the protrusions 261 may comprise substantially crescent shaped portions which are upstanding on the dispersion member 253, which straddle the apex of each of the ridges 257, with the inner face of the protrusion 261 facing towards the outward edge of the dispersion member 253. The approximate dimensions are 5 mm deep and 20 mm wide.

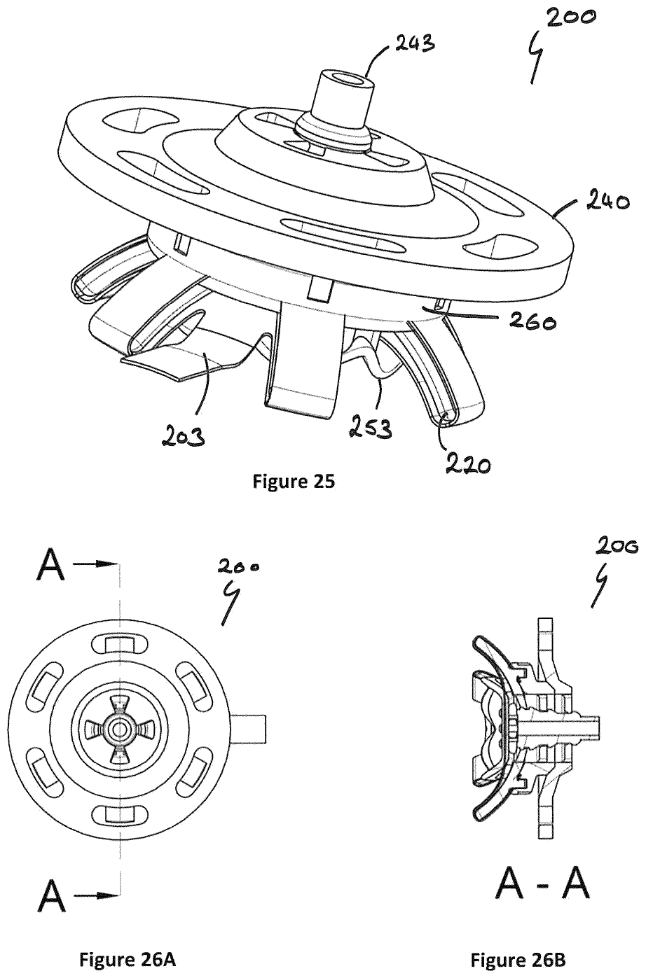

The alternative embodiment of the electrical measurement apparatus 200 may further comprise an attachment means 240 for removably coupling the electrical measurement apparatus 200 to a piece of headgear or the like (not shown). The attachment means 240 is typically coupled to the cover 260. The attachment means 240 defines a first part of a co-operable attachment means which is co-operable with a corresponding other second part (not shown) typically provided upon the headgear or the like to which the electrode 205 is to be coupled. The attachment means 240 preferably comprises a central aperture through which the retaining member 244 may extend when the attachment means 240, cover 260, support structure 220, flexible circuit 203 and dispersion member 253 are coupled together such as is shown in FIG. 25.

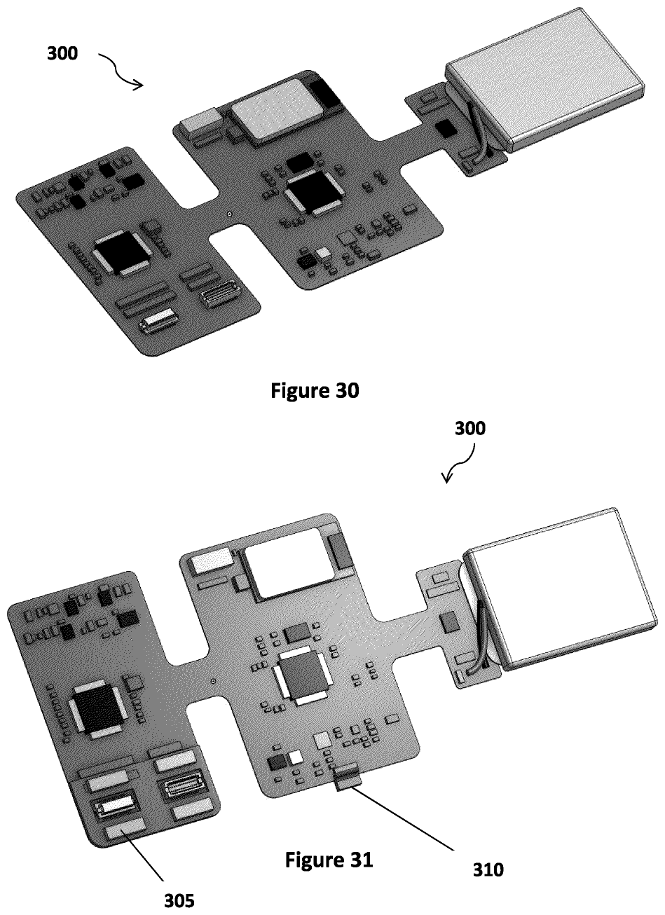

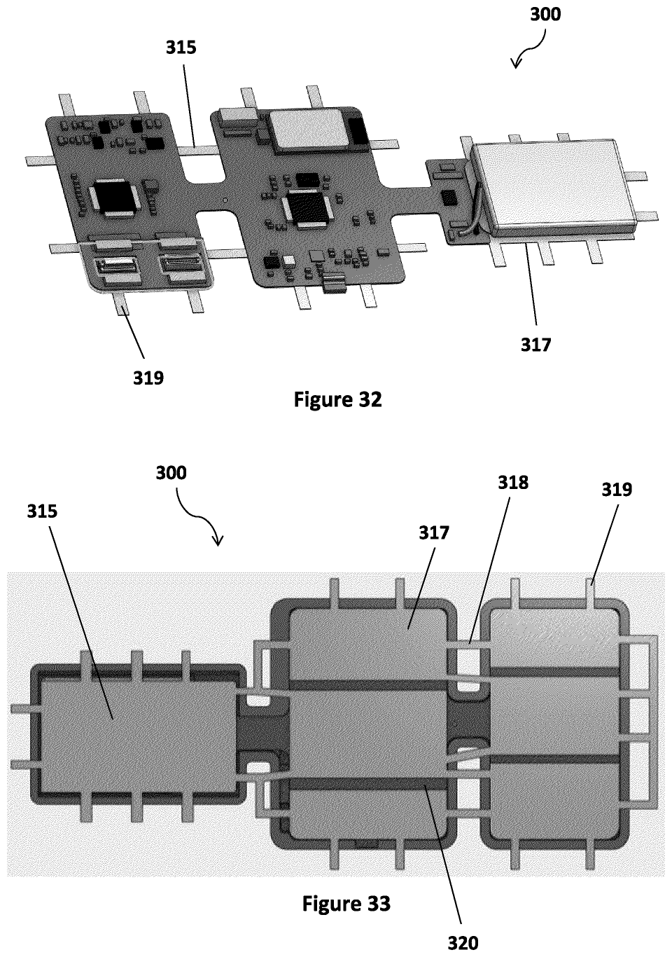

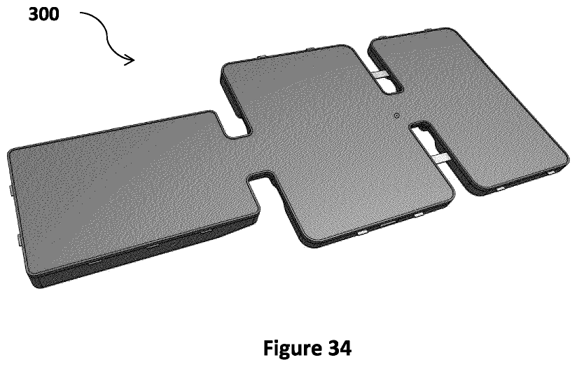

The flexible circuit 3 is typically protected by a protective means comprising a covering or housing or other suitable means for protecting the flexible circuit 3. In one embodiment the protective means may comprise a moulded covering or the like. In particular at least the posterior portion 11, where the majority of the processing circuitry is typically located, is typically protected by the protective means. In a preferred embodiment protective means comprises overmoulding the flexible circuit 3,300 with a plastic covering to provide protection to the circuit 3, 300 in-use. The method of overmoulding the flexible circuit 3 typically comprises the following steps:
Providing the flexible circuit 300, as shown in FIG. 30;
Masking or covering one or more areas of the flexible circuit 300, typically to prevent penetration by moulding materials into or onto unwanted areas of the flexible circuit 300;
Attaching, typically by bonding, one or more components to the flexible circuit 300, the one or more components may comprise magnets 305 and/or a light pipe 310, as shown in FIG. 31;
Attaching, typically by bonding, a stiffener 315 to a first side of the flexible circuit 3, typically the rear side thereof, the stiffener 315 providing structural support to the flexible support 3 during the overmoulding process, to this end the stiffener 315 is substantially rigid, as shown in FIG. 32;
The stiffener 31 typically comprises one or more flat portions 317 with a plurality of tabs 319 extending, spaced apart around the periphery of the one or more flat portions 317, the stiffener 315 further comprises one or more connecting portions 318 which extend between the flat portions 317 and couple at least two of the flat portions 317 together;
The stiffener tabs 319 aid in suspending the flexible circuit 3 in an tool during the overmoulding process;
In an alternative embodiment, shown in FIG. 33, the one or more flat portions 317 may comprise a plurality of separate portions which are typically joined together by one or more of the connecting portions 318, with the plurality of separate portions being spaced apart with respect to one another by a gap 320; Advantageously this provides for greater flexibility whilst retaining the enhanced structural integrity provided by the stiffener 315;
Overmoulding the flexible circuit 300 and coupled stiffener 315 in a plastic as shown in FIG. 34, wherein the plastic typically comprises polyurethane;
Optionally, one or more the tabs 319 may be trimmed if they are unnecessary; and
Further optionally a label or other sticker or the like may be applied to one or more surfaces of the overmoulded flexible circuit 300.

The invention is not limited to the embodiment(s) described herein but can be amended or modified without departing from the scope of the present invention.

1.-30. (canceled)

31. An electrical measurement apparatus, comprising:
a flexible circuit, the flexible circuit comprising electrical circuitry carried by a flexible substrate, said circuitry comprising one or more electrodes,
wherein said one or more electrodes comprise a central portion and a plurality of legs extending radially outwards from the central portion in a spaced apart relationship, wherein at least one electrical contact is located on each one of said plurality of legs; and
wherein said one or more electrodes are configured such that the plurality of electrical contacts of the one or more electrodes contact a user's head in use.

32. The electrical measurement apparatus of claim 31, wherein the flexible circuit further comprises processing circuitry to which the one or more electrodes are integrally connected.

33. The electrical measurement apparatus of claim 31, wherein said flexible circuit comprises a plurality of the electrodes; and/or wherein the electrical circuitry extends within the flexible substrate to tips of each of the plurality of legs of the electrode.

34. The electrical measurement apparatus of claim 31, wherein said electrodes comprise a support structure which is coupled to the central portion and/or one or more of the plurality of legs.

35. The electrical measurement apparatus of claim 34, wherein the support structure comprises a main body with a plurality of limbs extending radially outwards from the main body in a spaced apart relationship.

36. The electrical measurement apparatus of claim 35, wherein the support structure has a substantially domed shaped; and/or wherein the cross section of the support structure, extending from a distal end of a first limb to a distal end of an opposing second limb traversing the main body, is substantially arcuate.

37. The electrical measurement apparatus of claim 34, wherein the plurality of legs each comprise an aperture located at or towards a distal end of each leg.

38. The electrical measurement apparatus of claim 35, wherein protrusions are located at or towards the proximal ends of the plurality of limbs; and/or wherein the protrusions are located on a first surface of the support structure; and/or wherein the plurality of legs are folded laterally about the plurality of limbs, wherein the apertures of the plurality of legs align and engage with the plurality of protrusions to couple the plurality of legs to the support structure.

39. The electrical measurement apparatus of claim 38, wherein the central portion is located adjacent to or in contact with a second surface of the support structure when the plurality of legs are coupled to the support structure; or wherein the portion of each of the legs which is folded laterally about the tips of the limbs defines at least part of the electrical contact for each leg.

40. The electrical measurement apparatus of claim 35, wherein the support structure is coupled to the central portion and/or the one or more of the plurality of legs by an adhesive or heat-sealing.

41. The electrical measurement apparatus of claim 34, wherein said electrode further comprises a cover which is coupled to the support structure.

42. The electrical measurement apparatus of claim 41, wherein said cover is coupled to the first surface of the support structure.

43. The electrical measurement apparatus of claim 42, wherein said cover comprises a plurality of recesses on its underside which are configured to engage with the protrusions formed on the first surface of the main body such as to couple the cover to the support structure; or wherein the first surface of the main body comprises a central protrusion which is configured to engage with a co-operable recess provided on the underside of the cover such as to couple the cover to the support structure.

44. The electrical measurement apparatus of claim 31, wherein the central portion, the support structure and the cover each comprise a respective central aperture through which a retaining member comprising a pin is inserted to couple the support structure and cover or the support structure, cover and central portion together.

45. The electrical measurement apparatus of claim 32, wherein said processing circuitry is located in a portion of said flexible circuit that is spaced apart from the respective portions that carry said electrodes.

46. The electrical measurement apparatus of claim 35, wherein the support structure is made of rubber and/or plastic.

47. The electrical measurement apparatus of claim 31, further comprising means for receiving a fluid comprising a conductive gel or saline solution and a dispersion member that is configured to distribute the fluid received via the means for receiving the fluid towards the electrical contacts.

48. The electrical measurement apparatus of claim 47, wherein the means for receiving a fluid comprises a retaining member which is configured to couple the dispersion member and support structure together, wherein the retaining member comprises a hollow elongate member defining a substantially tube like structure and/or wherein the hollow elongate member defines a channel for receiving the fluid; and/or wherein the dispersion member comprises one or more outlets which are in fluid communication with the channel when the dispersion member is coupled to the retaining member; and/or wherein the dispersion member is shaped and dimensioned to direct fluid which egress from the outlets towards the electrical contacts.

49. A headgear apparatus comprising:
headgear; and
the electrical measurement apparatus as claimed in claim 31 coupled to the headgear.

50. A method of manufacturing the electrical measurement apparatus as claimed in claim 35, the method comprising:
providing the flexible circuit comprising electrical circuitry carried by the flexible substrate, said circuitry comprising the one or more electrodes;
coupling the support structure to the flexible circuit.
